(19) United States
(12) Patent Application Publication
(10) Pub. No.: US 2005/0163712 A1
(43) Pub. Date: Jul. 28, 2005

(54) Method for identifying bronchoconstriction relaxing substances

(76) Inventors: Staffan Skogvall, Lund (SE); Henrik Bjork, Lund (SE); Per Magnus Berglund, Lund (SE); Maria Fanny Dalence Guzman, Lund (SE); Olov Sterner, Malmo (SE)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/761,345

(22) Filed: Jan. 22, 2004

(51) Int. Cl. A61K 49/00; A61K 35/78; A61K 38/10; A61K 31/445; A61K 31/4172; A61K 31/225; A61K 31/557

(52) U.S. Cl. 424/9.2; 424/760; 514/2; 514/400; 514/317; 514/548; 514/642; 514/573

(57) ABSTRACT

In a method of measuring a relaxing effect on constricted human bronchi of a candidate substance a bronchus tissue preparation is mounted to a force transducer in a test apparatus. After conditioning the preparation is exposed for a contraction-effective dose of a known contraction-effective substance to make it assume a first tensioned state. The preparation is then exposed for a dose of the candidate substance to make it assume a second tensioned state. By comparing the contraction forces recorded for said tensioned states, a measure of the bronchorelaxing efficiency of the candidate substance is obtained. Also disclosed is a candidate substance thus identified, its uses, and a corresponding bronchus tissue preparation.

### FIELD OF THE INVENTION

The present invention relates to a method of identifying bronchoconstriction relaxing substances acting on a vanilloid receptor. The invention also relates to substances thus identified, pharmaceutical compositions comprising such substances, and their use as well as to the use of a bronchus preparation.

### BACKGROUND OF THE INVENTION

The vanilloid (“VR1”) receptor belongs to the large family of transient receptor potential cation channels (Gunthorpe et al, 2002) and is located mainly on thin unmyelinated pain fibers (C-fibers). The receptor functions as a molecular integrator of nociceptive stimuli, including heat, protons and various ligands, such as capsaicin and resiniferatoxin (Caterina et al. 1997). Several diseases have been associated with the vanilloid receptor, such as neuropathic pain, inflammatory bowel disease (Yiangou et al. 2001) and bladder diseases (Birder et al. 2002), and antagonists to this receptor has been proposed as potential new therapies against these diseases.

The first specific VR1 receptor antagonist capsazepine was discovered 1992 by Bevan et al. In that paper, the ability of the test substances to inhibit capsaicin-induced effects were evaluated in three rat tissue models. Capsazepine was found to be a reversible competitive antagonist of capsaicin with a moderate potency (Walpole et al 1994). In 1998, U.S. Pat. No. 5,840,720 described that 4-O and 5-aminomethylation of synthetic capsaicin derivatives provides new capsaicin receptor antagonist. The VR1 antagonistic effect of these receptor antagonist were evaluated in several models: inhibition of capsaicin-induced contractions in i) isolated guinea pig airway preparations, ii) guinea pig atrium preparations, and iii) guinea pig ileum preparations. In addition, the ability of the test substances to inhibit capsaicin-induced increase of blood pressure and heart rate was examined in rat in vivo experiments. The experiments showed that the substances were somewhat more potent than capsazepine in blocking the vanilloid receptor in the models. In 2001, Wahl et al. described that iodinization of the ultra-potent capsaicin analogue resiniferatoxin results in a VR1 antagonist that is at least 40 times more potent than capsazepine. The VR1 antagonistic effect was evaluated by the ability of iodo-resiniferatoxin (I-RTX) to inhibit capsaicin-induced effects in rodents.

Wahl et al. examined the VR1 blocking activity of the test substances in three in-vitro models; (1) capsaicin stimulation of calcium influx in cells expressing human VR1, (2) capsaicin-induced calcium influx in rat dorsal root ganglion cells and (3) capsaicin-induced rat bladder detrusor contractions. In addition, the effect of one of the compounds was examined on the capsaicin-induced overactive bladder in vivo model in anaesthetised rats.

Recently, several patent applications describing groups of substances with vanilloid receptor blocking properties have been published. WO 02/072536 A1 discloses urea derivatives having vanilloid receptor antagonist activities. The screen used for the compounds of that invention was derived from a FLIPR based calcium assay, as described by Smart et al. (2000). Compounds having antagonistic activity against human dorsal root ganglion neuron VR1 were identified by detecting differences in fluorescence after capsaicin addition in cells pre-incubated with test substance or buffer control. WO 02/076946 A2 discloses novel pyridine derivatives that are functional blockers of the human vanilloid receptor. Vanilloid receptor inhibition of the test agents was demonstrated by use of a fluorescence assay utilising calcium sensitive dyes to measure changes in calcium. The VR1 receptor was stimulated by application of either capsaicin or low pH. Cultures of Chinese hamster ovary cells expressing human VR1 receptor from dorsal root ganglion neurons were examined (McIntyre et al 2001). WO 02/090326 A1 describes new heterocyclic urea derivatives with vanilloid receptor antagonist activity. The effect of the test substances was evaluated as in WO 02/072536 A1.

WO 03/022809 describes novel urea derivatives with vanilloid receptor antagonistic properties. The blocking activity of the test substances was determined by a FLIPR based calcium assay, as in WO 02/072536. In addition, in-vivo experiments with FCA-induced hyperalgesia was used. WO 03/022809 A2 discloses new urea derivatives with vanilloid receptor blocking properties. The potency of the test substances was determined by using a FLIPR based calcium in vitro assay stimulated by capsaicin, similar to the methods used in WO 02/072536 A1. In addition, the ability of the test substances to inhibit FCA-induced hyperalgesia in the guinea pig was examined. WO 03/053945 A2 discloses new urea derivatives with vanilloid receptor antagonistic effect. Their efficiency was evaluated by a FLIPR based calcium assay, as in WO 02/072536 A1. WO 03/055484 discloses new urea derivatives with VR1 antagonistic effect. The VR1-blocking effect was examined by measurements of capsaicin-induced calcium influx in a CHO cell line transfected with human dorsal root ganglion vanilloid and P2X1 receptors. In addition, the effect of the test substances to inhibit capsaicin-induced bladder contraction in the rat was examined. WO 03/0555848 A2 discloses new urea derivatives with VR1 antagonistic properties. The effect of the test substances were examined as in WO 03/055484 and in the capsaicin-induced calcium influx model in primary cultured rat dorsal root ganglia neurons. WO 03/068749 A1 discloses new amides with VR1-blocking effects. The efficiency of the substances were tested in the FLIPR based calcium assay, where the ability to inhibit capsaicin-induced calcium influx was determined, and in the FCA-induced hyperalgesia model in guinea pig. WO 03/070247 describes fused azabicyclic compounds that inhibit the VR1 receptor. To test the potency of the substances, a FLIPR based model of capsaicin-induced calcium increase was determined. In addition, the mice antinociceptive test was performed.

WO 02/08221 discloses diaryl piperazines and related compounds with capsaicin receptor antagonistic properties. The vanilloid receptor antagonistic activity of the test substances was determined by their ability to inhibit capsaicin-induced calcium influx in human embryonic kidney cells transfected with expression plasmids for a human vanilloid receptor. WO 02/16317 A1 discloses novel thiocarbamic acid derivatives with antagonistic activity against the vanilloid receptor. The activity of test compounds was assayed in calcium influx studies and patch clamp tests on rat dorsal root ganglion nerve cells activated by capsaicin. In addition, some substances were evaluated for its analgesic and anti-inflammatory activity in rodents. WO 02/16318 A1 discloses novel thiourea derivatives with VR1 modulating properties. The VR1 blocking properties of the test substances were evaluated as in WO 02/16317. WO 02/16319 A1 discloses new thiourea compounds with vanilloid receptor antagonistic effects. The effect of the test substances was evaluated as in WO 02/16317 A1. WO 03/014064 A1 discloses amine derivatives with vanilloid receptor antagonistic activity. In 2003, Appendino et al published a paper describing that halogenation of a capsaicin analogue leads to novel vanilloid (“TRPV1”) receptor antagonists. The ability of these compounds to inhibit capsaicin-induced calcium mobilisation on recombinant human TRPV1 receptors (normally expressed in human dorsal root ganglia cells) over-expressed in human embryonic kidney cells was investigated (Hayes et al. 2000). The best of the test substances was also tested on native TRPV1 in: 1. rat dorsal root ganglion neurons; 2. guinea pig urinary bladder; and 3. guinea pig bronchi. This substance was significantly more potent in regard of vanilloid blocking properties than capsazepine in tissues 1-2, but not in the guinea pig bronchi.

There are problems with present test models. It is well established that the vanilloid receptor displays a marked interspecies difference (Szallasi 1994, Szallasi et al. 1999, McIntyre et al. 2001). One striking example of this is that the dose of capsaicin that can kill the guinea pig almost instantly is well tolerated by the hamster (Glinsukon et al. 1980). Guinea pig airway preparations contract strongly when exposed to capsaicin (Lundberg et al. 1987, Djokic et al. 1989), and this contraction is mediated by a release of the tachykinins substance P and neurokinin A from sensory C fibers. This high sensitivity of the guinea pig to capsaicin is also found in in vivo experiments, where capsaicin exposure can give a lethal bronchoconstriction. In contrast, human bronchial preparations display only weak contractions, or even relaxations, when exposed to capsaicin (Spina et al 1998). This low sensitivity in human airways to capsaicin is also demonstrated by the fact that healthy humans that inhale capsaicin normally do not experience any significant bronchoconstriction, only cough (Hathaway et al, 1993). Humans and animals also differ considerably in regard of pharmacological sensitivity to vanilloid receptor blocking substances. Undem and Kollarik (2002) reported that I-RTX inhibits capsaicin-induced bronchoconstriction in guinea pig airway preparations with a potency 10-30 times higher than capsazepine. In contrast to capsazepine, the present inventors found that I-RTX was did not inhibit leukotriene-induced bronchoconstriction in human bronchial preparations.

There are also reports of variation in pharmacological sensitivity to VR1 modulating substances in different tissues from the same animal. Szallasi (1994) described such variation to capsaicin and RTX in central and peripheral vanilloid receptors, as well as diverging sensitivity to capsaicin and capsazepine in urinary bladder tissue compared to tissues from the airways and colon. Appendino et al (2003) found that a new capsaicin analogue was a more potent vanilloid receptor antagonist than capsazepine in rat dorsal ganglion neuron cells and guinea pig urinary bladder tissue but not in guinea pig bronchial preparations.

### OBJECTS OF THE INVENTION

It is an object of the invention to provide an improved method of identifying bronchoconstriction relaxing substances acting on a vanilloid receptor.

Further objects of the invention will become apparent from the following short summary of the invention, the description of preferred embodiments thereof illustrated in a drawing, and the appended claims.

### SUMMARY OF THE INVENTION

The present invention is based on the insight that, in the evaluation of substances that are candidates for bronchoconstriction-relaxing drugs capable of inhibiting the vanilloid receptor in the airways of humans, it is vital that preparations of relevant origin and relevant tissues (airway preparations, in particular bronchi) are used. The use of human airway preparations is however dissuaded from by the fact that they are rather insensitive to capsaicin. The reason for the weak bronchial effects by capsaicin in human airways is unclear but, in the opinion of the present inventors, might be explained by the presence of a different subtype of the VR1 receptor in human airway smooth muscle tissue. According to this hypothesis, which is however not binding and given for tentative explanation only and does not affect the working of the invention, the VR1 receptor is highly sensitive to activation by endogenous substances, such as the arachidonic acid product leukotriene, whereas it exhibits only low sensitivity to capsaicin. The fact that capsaicin inhalation does provoke cough in humans does not contradict this; a possible explanation is that this is caused by activation of other types of (non-VR1) airway receptors or, alternatively, by different, capsaicin-sensitive VR1 receptor subtypes in the upper airways.

According to the present invention, a model was developed to conveniently determine the VR1 blocking properties of candidate substances, that is, substances selected for screening of their VR1 blocking properties. The model comprises in-vitro examination of the force development in human isolated airway preparations mounted in experimental chambers exposed to vanilloid receptor activating (contracting) substances during control and test conditions. The suppression of the activating effect of the endogenous transmitter by a candidate (test) substance is a measure of its VR1 blocking effect. It is vital to use human airway preparations due to the inter- and intra-species differences of the vanilloid receptor, but the use of human airway preparations is complicated by the fact that the standard drug for activation of the VR1 receptor, capsaicin, does not produce clear, reproducible contractions in human airway preparations. According to the present invention, it is assumed that inflammatory mediators mainly contract airway smooth muscle indirectly by activation of the pre-synaptic vanilloid receptor. This stimulates the C-fibers to release transmitters that contract the smooth muscle fibers. An alternative explanation is that the relevant vanilloid receptors are located directly on the smooth muscle fibers.

In particular, according to the present invention, is disclosed a method of measuring a bronchorelaxing effect on constricted bronchi, of a candidate substance, the effect possibly being caused by the action thereof on a vanilloid (VR1) receptor in the bronchi, wherein the method comprises: (a) providing a bronchus tissue preparation; (b) mounting the preparation immersed in a physiological medium in an apparatus for determining its contractile state, the apparatus comprising a force transducer fixed to the preparation by means of which the contractile state of the preparation is recorded; (c) conditioning the preparation to establish a substantially non-tensioned base line state; (d) exposing the preparation for a contraction-effective dose of a known contraction-effective agent or a contraction-effective electrical field to make it assume a first tensioned state;(e) exposing the preparation for the candidate substance; (f) allowing the preparation to return to a base line state; (g) repeating step (d) to make the preparation assume a second tensioned state; (h) comparing the contraction recorded from the respective tensioned state in steps (d) and (g) to the base line state to obtain a measure of the bronchorelaxing efficiency of the candidate substance; (i) optionally comparing said measure of bronchorelaxing efficiency with that obtained with capsazepine or other known VR1 antagonist. In this specification “bronchorelaxing” refers to “broncho-constriction relaxing”. The exposition of the preparation to the candidate substance in step (e) is preferably consecutive to the exposition to the known contraction-effective agent in step (d) but substantially simultaneous with the exposition to the known contraction-effective agent in step (g). The time period from the first contraction maximum to the second contraction maximum is preferably one hour or more.

It is preferred for the known broncho-constrictive substance to be selected from leukotriene D4, prostaglandin, histamine, cytokine, acetylcholine, in particular to be leukotriene D4.

It is preferred for the physiological medium to be a physiological saline solution (PSS) comprising one or several of Na⁺, K⁺, Mg²⁺, Ca²⁺, SO₄²⁻, HCO₃⁻, Cl⁻, glucose.

According to a preferred aspect of the invention the contraction-effective dose of a known broncho-constrictive substance is one that is capable of eliciting a contraction force of 100 mg or more, in particular from 200 to 500 mg.

According to another preferred aspect of the invention is disclosed a substance having a relaxing effect on constricted human bronchi by affecting a vanilloid (VR1) receptor in the bronchi, which substance has been identified as a bronchoconstriction relaxing agent by the method of the invention. The substance so identified may be advantageously be used for the manufacture of a medicament for treating a broncho-constrictive condition caused by a vanilloid (VR1) receptor agonist and in a method of treating a broncho-constrictive condition in a person caused by a vanilloid (VR1) receptor agonist comprising its administration to that person in a dose, which is effective in relaxing bronchoconstriction. According to the present invention is also disclosed the use of a human bronchus tissue preparation for assessment of the broncho-relaxing activity of a candidate substance in regard of a vanilloid (VR1) receptor.

Instead of human bronchi, other human airway tissue, in particular tissue from the trachea, may be used in the present invention.

The invention will now be explained in greater detail by reference to a number of figures illustrating preferred but not limiting embodiments of the invention.

### SHORT DESCRIPTION OF THE FIGURES

FIG. 1 is a time v. force diagram of the determination of the bronchoconstriction relaxing effect of capsazepine. At (B) the preparation is mechanically tensioned by a selected force;

FIGS. 2-4 illustrate relevant sections of corresponding time v. force diagrams for three candidate substances.

### DESCRIPTION OF PREFERRED EMBODIMENTS

Materials and Apparatus

Dissection and mounting of lung tissue preparations. Lung tissue was obtained from patients undergoing lobectomia or pulmectomia due to lung carcinoma. The tissue was placed in a dissection chamber continuously perfused with 10 ml min⁻¹ of a physiological saline solution (PSS) at room temperature. An airway was identified in the cut part of the lobe, and a bronchus of 10-20 mm length and 1-2 mm diameter was obtained. The bronchus was cut into rings of a width of about 2-3 mm. Each bronchial ring was cleaved to obtain an about rectangular oblong preparation, one end of which was tied to a small steel hook connected to a force transducer, while the other end of the preparation was attached to a fixed hook. This is followed by a period of adjustment, as described below. The preparation was mounted in an atmosphere containing 12% of oxygen and 6% of CO₂.

Experimental chamber. The experimental chamber has a a volume of 5 ml. It is perfused with PSS at a rate of 3 ml min⁻¹. Two preparations are mounted in the chamber, and measurements on them are performed in parallel. For mechanical tensioning each force transducer (AME 801, SensoNor A/S, Horten, Norway) is connected to a micrometer screw. The substances to be tested, the reference substance (capsazepine), and transmitter (LTD4) are injected upstream of the preparation(s).

Materials. PPS (physiological saline solution, in mM): NaCl, 117; KCl, 4.87; MgSO₄, 0.60; NaHCO₃, 25.0; CaCl₂, 1.60; glucose, 5.23. The solution is saturated with a mixture of 94% oxygen and 6% carbon dioxide, giving a pH of 7.40±0.05 in the experimental chamber. All substances are prepared as stock solution dissolved in the vehicles ethanol or DMSO. Leukotriene D4 (LTD4; Keyman Ltd.): 10 μl of a 100 μM ethanol stock solution. Capsazepine (Sigma Aldrich): 10 μl of a 0.1 M ethanol stock solution. Substance to be tested: 10-100 μl of a 0.01-0.1 M ethanol or DMSO stock solution. Solution for establishing the passive tension level: calcium-free PSS+2 mM EGTA+20 mM caffeine. To exclude effects by the test substance vehicle, ethanol or DMSO, respectively, were added during the entire experiment except during the presence of test substance.

### EXAMPLE 1

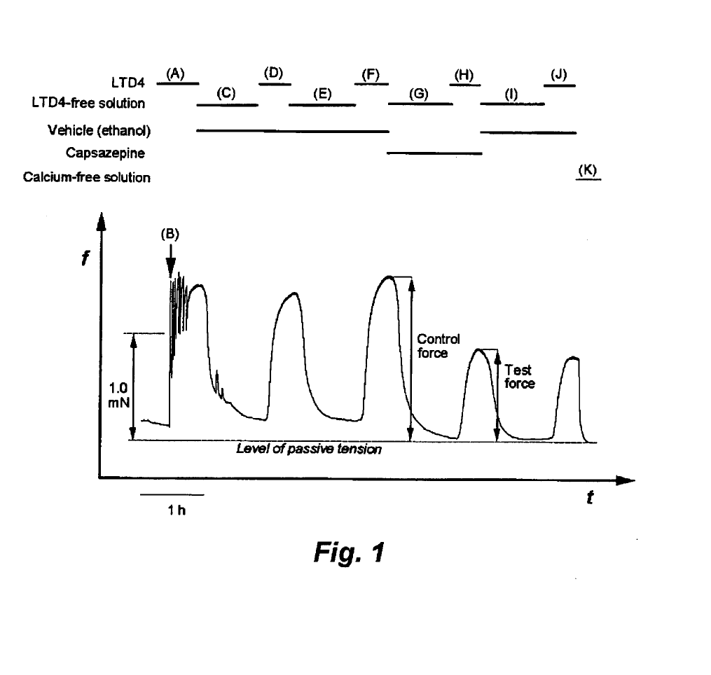

Test procedure. An exemplary test is shown in FIG. 1 in which capital letters indicate interference with the test system. The material for the preparation was a bronchus (inner diameter about 1 mm) from a male occasional smoker (41 yrs) but with the epithelium intact. Adjustment and stretch. After mounting as described above the preparation is allowed to adjust with a low passive tone in the experimental chamber. The composition of the gas is changed to 94% (v/v) of oxygen. After a short adjustment period, PSS with 10 nM LTD4 is added to the experimental chamber upstream of the preparation (A). The preparation is stretched repeatedly (B) until it exerts a contraction force of around 150 mg. When the contraction has levelled off, leukotriene-free solution is administered for 1 hour (C), resulting in a relaxation. A second injection of 10 nM LTD4 (D) makes the preparation return to the tensioned state. At the peak tension leukotriene-free solution is again administered (E). After a third injection of 10 nM LTD4 (F) the preparation returns to the tensioned state. At the peak, PSS with 10 μM capsazepine (G) is added, resulting in a relaxation. After 1 h exposure to capsazepine, LTD4 is added, resulting in a contraction (H). In comparison with the control LTD4 contraction (F), a substantially weaker contraction is now observed (H). To obtain a measure of the test substance's bronchorelaxing effect the test and control forces registered in the experiment are compared. In the present experiment a remaining contraction (test force) of about 55% of that caused by the control force was registered. After allowing one hour for return to baseline conditions (I) 10 nM LTD4 is again injected (J) to determine the reversibility of the VR1 receptor inhibition. During steps C-F and I-J 10 μl ethanol per 100 ml PSS is present to compensate for potential vehicle effects. The experiment is concluded by adding calcium-free solution with addition of 2 mM EGTA and 20 mM caffeine for 20 min to establish the passive tension level (K).

A VR1 receptor antagonist candidate can be tested for antagonist properties by substituting capsazepine for it in the test system. A measure for its blocking capacity is obtained by comparing the result (% blocking of contraction by LTD4) with that obtained with capsazepine. If the remaining contraction after exposure to a test substance is larger than after exposure to capsazepine, the test substance is less effective than capsazepine in regard of VR1-blocking properties. If, on the other hand, the remaining contraction after exposure to a test substance is smaller than after exposure to capsazepine, the test substance is more effective than capsazepine in regard of VR1-blocking properties. Instead of capsazepine, any other suitable substance can be used as a standard for comparing VR1-blocking properties. A preparation is considered stable and thus fit for the evaluation of test substances if the difference in contraction between contractions D and F is less than 15 per cent.

It should be noted that, although LTD4 is normally used to contract the preparations, it is possible to use any other suitable transmitter or substance with a broncho-constrictive effect. Examples of useful broncho-constrictive substances that may be used instead of LTD4 include cholinergic receptor agonists such as acetylcholine, charbacholine, metacholine and other M3-agonists; adenosine receptor agonists; bombesin receptor agonists; bradykinin receptor agonists; cannabinoid receptor agonists; chemokine receptor agonists; cytokine receptor agonists; dopamine receptor agonists; glutamate receptor agonists; glycine receptor agonists; high concentrations of potassium chloride; histamine receptor agonists such as histamine and other H1-agonists; leukotriene receptor agonists; neuropeptide Y receptor agonists such as neuropeptide Y; opioid receptor agonists such as fentanyl; platelet activating factor receptor agonists such as platelet activating factor (PAF); prostanoid receptor agonists such as prostaglandin F2-alpha, other prostaglandins, tromboxane A2; tachykinin receptor agonists such as neurokinin A, neurokinin B, substance P.

It is also possible to contract the preparation by stimulating it by applying an electrical field (EFS). Another possibility is to examine the relaxing effects by test substances on airway preparations displaying a spontaneous tension development, a so-called spontaneous tone.

### EXAMPLE 2

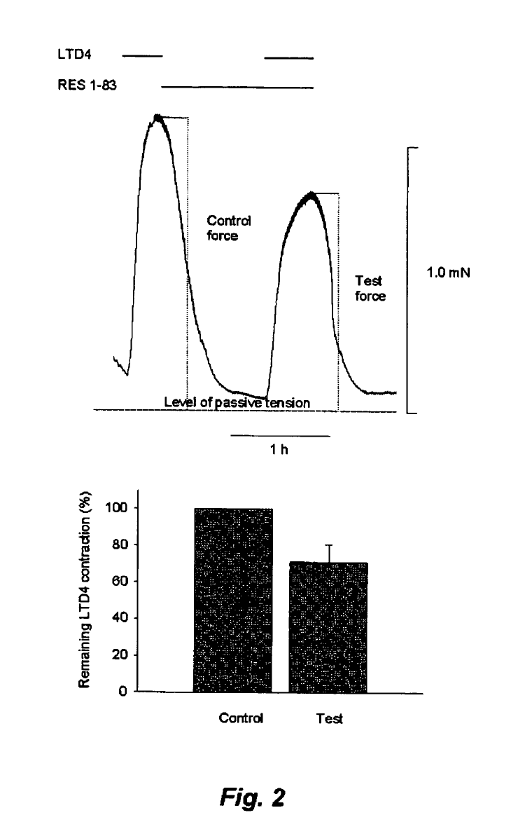

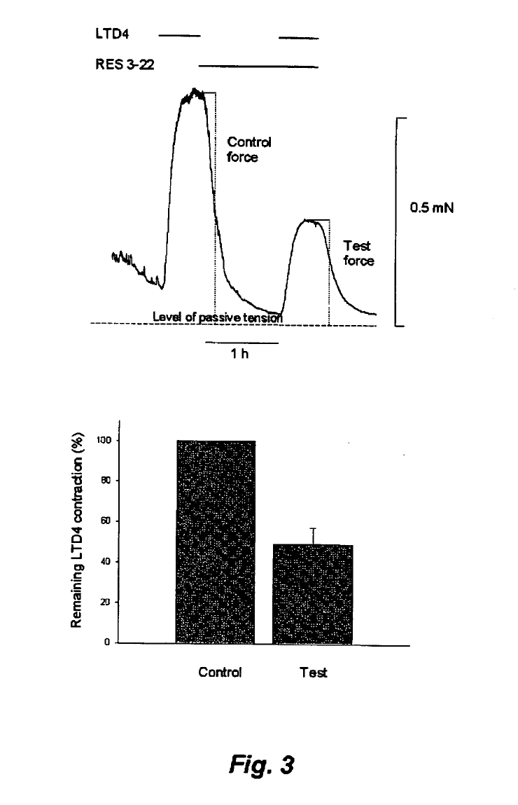

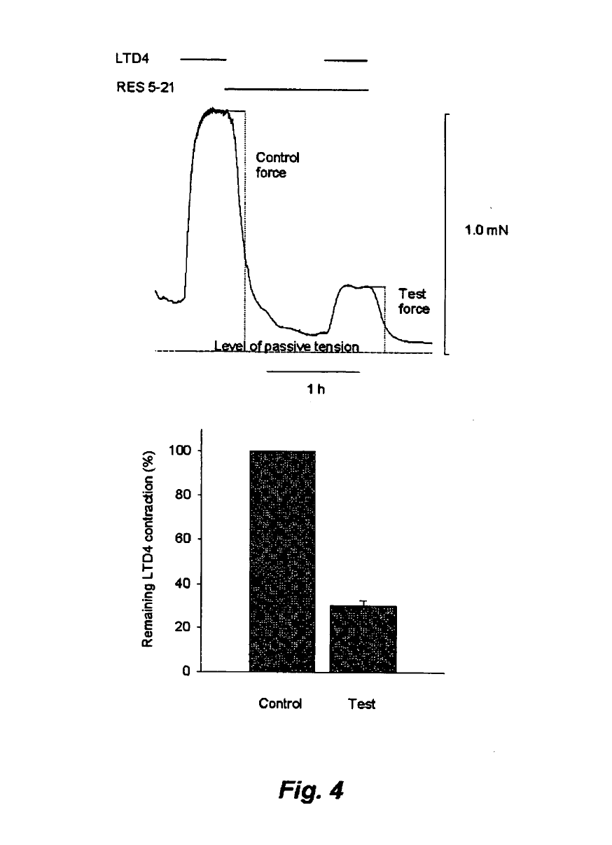

Three synthetically obtained compounds designated 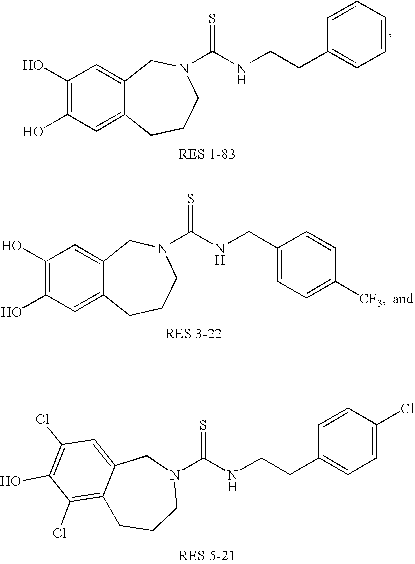
were tested for broncho-relaxing effect in the test system of Example 1. Relevant sections of the recorded force v. time diagrams are shown in FIGS. 2 to 4. LTD4 was used as a broncho-contracting agent. The three compounds were all found to exhibit a broncho-contracting effect. In comparison to capsazepine (FIG. 1) 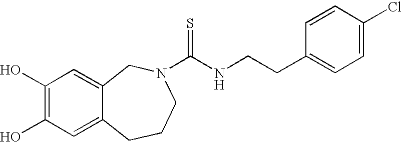
RES 1-83 (FIG. 2) was found to be a substantially less effective, compound RES 3-22 to be about equally effective, and compound RES 5-21 to be substantially more effective in relaxing broncho-constriction.

### REFERENCES

Appendino G et al., Br J Pharmacol. 2003; 139(8): 1417-1424. Halogenation of a capsaicin analogue leads to novel vanilloid TRPV1 receptor antagonists.

Bevan S et al., Br J Pharmacol. 1992; 107(2): 544-52. Capsazepine: a competitive antagonist of the sensory neurone excitant capsaicin.

Birder L A et al., Nat Neurosci. 2002; 5(9): 856-60. Altered urinary bladder function in mice lacking the vanilloid receptor TRPV1.

Brink C et al., Br J Pharmacol. 1980; 69(3): 383-8. The interaction between indomethacin and contractile agents on human isolated airway muscle.

Caterina M et al., Nature 1997; 389(6653): 816-24. The capsaicin receptor: a heat-activated ion channel in the pain pathway.

Dahlen S E et al., Acta Physiol Scand. 1983; 118(4): 393-403. Mechanisms of leukotriene-induced contractions of guinea pig airways: leukotriene C4 has a potent direct action whereas leukotriene B4 acts indirectly.

Djokic T D, J Pharmacol Exp Ther. 1989; 248(1): 7-11. Inhibitors of neutral endopeptidase potentiate electrically and capsaicin-induced noncholinergic contraction in guinea pig bronchi.

Glinsukon T et al., Toxicon. 1980; 18(2): 215-20. Acute toxicity of capsaicin in several animal species.

Gunthorpe M J et al., Trends Pharmacol Sci. 2002; 23(4): 183-91. The diversity in the vanilloid (TRPV) receptor family of ion channels.

Hathaway T J et al., Am Rev Respir Dis. 1993; 148(5): 1233-7. Effects of inhaled capsaicin in heart-lung transplant patients and asthmatic subjects.

Hayes P et al., Pain. 2000; 88(2): 205-15. Cloning and functional expression of a human orthologue of rat vanilloid receptor-1.

Lundberg et al., Annu Rev Physiol. 1987; 49:557-72. Polypeptide-containing neurons in airway smooth muscle.

McIntyre P et al., Br J Pharmacol. 2001; 132(5): 1084-94. Pharmacological differences between the human and rat vanilloid receptor 1 (VR1).

Smart D et al., Br J Pharmacol. 2000; 129(2): 227-30. The endogenous lipid anandamide is a full agonist at the human vanilloid receptor (hVR1).

Spina D et al., Life Sci. 1998; 63(18): 1629-42. A comparison of sensory nerve function in human, guinea-pig, rabbit and marmoset airways.

Szallasi A, Gen Pharmacol. 1994; 25(2): 223-43. The vanilloid (capsaicin) receptor: receptor types and species differences.

Szallasi A et al., Pharmacol Rev. 1999; 51(2): 159-212. Vanilloid (Capsaicin) receptors and mechanisms.

Undem B J et al., J Auton Nerv Syst. 1993; 44(1): 35-44. Immunologically induced neuromodulation of guinea pig nodose ganglion neurons.

Undem B J and Kollarik M, J Pharmacol Exp Ther. 2002; 303(2): 716-22. Characterization of the vanilloid receptor 1 antagonist iodo-resiniferatoxin on the afferent and efferent function of vagal sensory C-fibers.

Walpole C S et al., J Med Chem. 1994; 37(13): 1942-54. The discovery of capsazepine, the first competitive antagonist of the sensory neuron excitants capsaicin and resiniferatoxin.

Wahl P et al., Mol Pharmacol. 2001; 59(1): 9-15. Iodo-resiniferatoxin, a new potent vanilloid receptor antagonist.

Yiangou Y et al., Lancet 2001; 357(9265): 1338-9. Vanilloid receptor 1 immunoreactivity in inflamed human bowel.

1. A method of measuring a bronchorelaxing effect of a candidate substance on constricted human bronchi, said effect being caused by action thereof on a vanilloid (VR1) receptor in the bronchi, comprising
(a) providing an apparatus for determining the contractile state of a bronchus tissue preparation having the preparation immersed in a physiological medium mounted therein, the apparatus comprising a force transducer fixed to the preparation;
(b) establishing a substantially non-tensioned base line state of the preparation;
(c) exposing the preparation for a contraction-effective dose of a contraction-effective agent to make it assume a first tensioned state;
(d) exposing the preparation to the candidate substance;
(e) allowing the preparation to return to a base line state;
(f) repeating step (c) to make the preparation assume a second tensioned state; and
(g) comparing the contraction maxima from the respective tensioned state in steps (c) and (f) to the baseline state to obtain a measure of the bronchorelaxing efficiency of the candidate substance.

2. The method of claim 1, wherein said exposition to the candidate substance is consecutive to the first exposition to the contraction-effective agent and simultaneous with said second exposition to the contraction-effective agent.

3. The method of claim 2, further comprising comparing said measure of bronchorelaxing efficiency with that obtained with a known VR1 antagonist.

4. The method of claim 2, wherein the broncho-constrictive agent is selected from the group consisting of LTD4; cholinergic receptor agonist; adenosine receptor agonist; bombesin receptor agonist; bradykinin receptor agonist; cannabinoid receptor agonist; chemokine receptor agonist; cytokine receptor agonist; dopamine receptor agonist; glutamate receptor agonist; glycine receptor agonist; high concentration of potassium chloride; histamine receptor agonist; leukotriene receptor agonist; neuropeptide Y receptor agonist; opioid receptor agonist; platelet activating factor receptor agonist; prostanoid receptor agonist, prostaglandin, tromboxane A2; and tachykinin receptor agonist.

5. The method of claim 4, wherein the broncho-constrictive substance is leukotriene D4.

6. The method of claim 4, wherein the broncho-constrictive agent is selected from the group consisting of acetylcholine, charbacholine, metacholine, histamine, neuropeptide Y, fentanyl, platelet activating factor (PAF), prostaglandin F2-alpha, neurokinin A, neurokinin B, and substance P.

7. The method of claim 2, wherein the broncho-constrictive agent is a contraction-effective electrical field.

8. The method of claim 1, further comprising comparing said measure of bronchorelaxing efficiency with that obtained with a known VR1 antagonist.

9. The method of claim 1, wherein the broncho-constrictive agent is selected from the group consisting of LTD4; cholinergic receptor agonist; adenosine receptor agonist; bombesin receptor agonist; bradykinin receptor agonist; cannabinoid receptor agonist; chemokine receptor agonist; cytokine receptor agonist; dopamine receptor agonist; glutamate receptor agonist; glycine receptor agonist; high concentration of potassium chloride; histamine receptor agonist; leukotriene receptor agonist; neuropeptide Y receptor agonist; opioid receptor agonist; platelet activating factor receptor agonist; prostanoid receptor agonist, prostaglandin, tromboxane A2; and tachykinin receptor agonist.

10. The method of claim 9, wherein the broncho-constrictive substance is leukotriene D4.

11. The method of claim 10, wherein the physiological medium comprises physiological saline solution and said contraction-effective dose is capable of eliciting a contraction force of at least 100 mg.

12. The method of claim 9, wherein the broncho-constrictive agent is selected from the group consisting of acetylcholine, charbacholine, metacholine, histamine, neuropeptide Y, fentanyl, platelet activating factor (PAF), prostaglandin F2-alpha, neurokinin A, neurokinin B, and substance P.

13. The method of claim 1, wherein the broncho-constrictive agent is a contraction-effective electrical field.

14. The method of claim 1, wherein the physiological medium is physiological saline solution (PSS) comprising at least one of Na⁺, K⁺, Mg²⁺, Ca²⁺, SO₄²⁻, HCO₃⁻, Cl⁻, and glucose.

15. The method of claim 1, wherein said contraction-effective dose is capable of eliciting a contraction force of at least 100 mg.

16. The method of claim 15, wherein said contraction force is about from 200 to 500 mg.

17. The method of claim 1, wherein the time period from said first contraction maximum to said second contraction maximum is at least one hour.

18. The method of claim 2, wherein the physiological medium comprises physiological saline solution and said contraction-effective dose is capable of eliciting a contraction force of at least 100 mg.

19. A substance having a relaxing effect on constricted human bronchi by effecting a vanilloid (VR1) receptor in the bronchi identified by the method of claim 1.

20. A method of treating a broncho-constrictive condition in a person caused by a vanilloid (VR1) receptor agonist comprising the administration to said person of a constricted bronchi relaxing-effective dose of the substance of claim 19.
